(19) United States
(12) Patent Application Publication
(10) Pub. No.: US 2024/0294724 A1
(43) Pub. Date: Sep. 5, 2024

(54) RECOVERING DI- AND/OR POLYISOCYANATES FROM PU-DEPOLYMERISATION PROCESSES

(71) Applicant: Evonik Operations GmbH, Essen (DE)

(72) Inventors: Ralph Marquardt, Frankfurt/Main (DE); Roland Hubel, Essen (DE); Annegret Terheiden, Alpen (DE); Felix Mühlhaus, Essen (DE)

(73) Assignee: Evonik Operations GmbH, Essen (DE)

(21) Appl. No.: 18/575,871

(22) PCT Filed: Jun. 28, 2022
(86) PCT No.: PCT/EP2022/067705

§ 371 Date: Jan. 1, 2024

(30) Foreign Application Priority Data
Jul. 2, 2021 (EP) 21183452.8

(51) Int. Cl. C08J 11/14; C08G 18/76; C08J 9/228

(52) U.S. Cl. CPC C08J 11/14; C08G 18/7621; C08J 9/228; C08J 2375/04

(57) ABSTRACT

A process is developed for producing aromatic and/or aliphatic di- and/or polyisocyanates derived from the depolymerization of a polyurethane by hydrolysis. A composition suitable for producing polyurethane foam is made. A process is also developed for the production of polyurethane using at least one recycled isocyanate component, and a polyurethane foam is made. A process is made for manufacturing goods where the polyurethane foam is insulation, cushioning, and other similar products.

The present invention is in the field of di- and/or polyisocyanates, of polyurethanes and of polyurethane recycling. In particular, a process for producing aromatic and/or aliphatic di- and/or polyisocyanates which derives from depolymerization of a polyurethane by hydrolysis is described.

On account of their exceptional mechanical and physical properties, polyurethanes find use in a very wide variety of sectors A particularly important market for a very wide variety of polyurethanes is the polyurethane foams sector. Polyurethanes (PU) are for the purposes of the present invention all reaction products derived from isocyanates, in particular from polyisocyanates, and appropriately isocyanate-reactive molecules. They also include polyisocyanurates, polyureas and isocyanate or polyisocyanate reaction products containing allophanates, biurets, uretdiones, uretonimines or carbodiimides.

Polyurethanes are now so widespread worldwide that recycling is becoming increasingly important for these materials too. Various decomposition processes for recovery of polyurethane wastes therefore already exist in the prior art. The known chemical processes such as hydrolysis, for example described in U.S. Pat. No. 5,208,379, glycolysis, acidolysis, aminolysis, hydrogenolysis, solvolysis and similar processes seek to effect depolymerization at a molecular level Such polyurethane decomposition processes also generate amines.

There is a fundamental need to optimally recover such amines which result from polyurethane decomposition processes. Key products are in particular the di- and/or polyamines resulting from the di- and/or polyisocyanates employed during polyurethane production (for example toluenediamine in the case of PU from toluene diisocyanate), which are to be recycled into the value cycle through workup and conversion into isocyanates. The use of such di- and/or polyamines to obtain aromatic and/or aliphatic di- and/or polyisocyanates having a sufficiently high quality to allow reuse in renewed polyurethane production is a particular aspiration. Realizing this is an object of the present invention.

This object is achieved by the subject matter of the invention. The invention provides a process for producing aromatic and/or aliphatic di- and/or polyisocyanates comprising the steps of
a) depolymerizing a polyurethane by hydrolysis in the presence of a base and a catalyst selected from the group consisting of quaternary ammonium salts containing an ammonium cation comprising 6 to 30 carbon atoms and organic sulfonates containing at least 7 carbon atoms at temperatures preferably below 200° C. to produce di- and/or polyamines, preferably comprising toluenediamine,
b) separating the di- and/or polyamines obtained from step a), preferably comprising toluenediamine, from the reaction mixture by extraction, distillation and/or other separation processes,
c) phosgenating the di- and/or polyamines obtained from step b) to afford di- and/or polyisocyanates, wherein di- and/or polyamines not deriving from process step a) may optionally also be added in the phosgenation step c).

The process makes it possible to provide aromatic and/or aliphatic di- and/or polyisocyanates such as in particular toluene 2,4-diisocyanate and toluene 2,6-diisocyanate which may in turn be used for renewed production of polyurethane foam, in fact with a reduced content of dichlorobenzene compared to standard PU foam. The energy-intensive alkylation, nitration and hydrogenation of benzene normally necessary to arrive at toluene 2,4-isocyanate for example are avoided. The invention moreover develops polyurethane wastes as a renewable raw material source for di- and/or polyisocyanates.

Conventional isocyanates are produced in large amounts and serve predominantly as starting materials for producing polyurethanes. Their production is usually carried out by reaction of the corresponding amines obtainable by upgrading of petrochemical raw materials with phosgene, wherein phosgene is employed in stoichiometric excess. The reaction of the amines with the phosgene may be carried out either in the gas phase or in the liquid phase, and the reaction may be performed discontinuously or continuously (W. Siefken, Liebigs Ann. 562, 75-106 (1949)). Processes for producing organic isocyanates from primary amines and phosgene have been extensively described in the prior art, see for example Ullmanns Encyklopädie der technischen Chemie, 4th Ed. (1977), Volume 13, pp. 351 to 353 and G. Wegener et al. Applied Catalysis A: General 221 (2001), pp. 303-335, Elsevier Science B.V. Isocyanates employed worldwide include not only aromatic isocyanates such as for example methylenediphenyl diisocyanate (MDI—“monomeric MDI”), polymethylene polyphenylene polyisocyanate (a mixture of MDI and higher homologues, PMDI, “polymeric MDI”) or toluene diisocyanate (TDI), but also aliphatic isocyanates such as for example hexamethylene diisocyanate (HDI) or isophorone diisocyanate (IPDI).

In a preferred embodiment of the invention, the resulting isocyanates comprise aromatic and/or aliphatic di- and/or polyisocyanates, for example methylenediphenyl diisocyanate (MDI-“monomeric MDI”), polymethylene polyphenylene polyisocyanate (a mixture of MDI and higher homologues, PMDI, “polymeric MDI”), toluene diisocyanate (TDI) and/or isophorone diisocyanate (IPDI), in particular 2,4′-diphenylmethane diisocyanate and/or 2,2′-diphenylmethane diisocyanate and/or polyphenyl polymethylene polyisocyanate (crude MDI) and/or 2,4-toluene diisocyanate and/or 2,6-toluene diisocyanate. 2,4-Toluene diisocyanate and/or 2,6-toluene diisocyanate are most preferred.

The present invention comprises depolymerizing a polyurethane by hydrolysis in the presence of a base and a catalyst selected from the group consisting of quaternium ammonium salts containing an ammonium cation comprising 6 to 30 carbon atoms and organic sulfonates containing at least 7 carbon atoms at temperatures preferably below 200° C. to produce di- and/or polyamines.

Corresponding and preferred hydrolysis processes of PU materials are for example described in the as yet unpublished European patent applications under filing numbers 20192354.7 or 20192364.6.

A particularly preferred variant, referred to here as preferred variant 1, of depolymerization by hydrolysis is described below.

In particular, it is preferable when the depolymerization of the polyurethane in step a) is effected using a base having a pk_{b} at 25° C. of 1 to 10, preferably 1 to 8, more preferably 1 to 7, in particular 1.5 to 6, and also a catalyst selected from the group consisting of (i) quaternary ammonium salts containing an ammonium cation comprising 6 to 30 carbon atoms and (ii) organic sulfonate containing at least 7 carbon atoms. This is a preferred embodiment of the invention.

Preferred bases contain an alkali metal cation and/or an ammonium cation. Preferred bases are alkali metal phosphates, alkali metal hydrogenphosphates, alkali metal carbonates, alkali metal silicates, alkali metal hydrogencarbonates, alkali metal acetates, alkali metal sulfites, ammonium hydroxides or mixtures of the above. Preferred alkali metals are Na, K or Li or mixtures of the above, in particular Na or K or mixtures thereof, preferred ammonium cation is NH₄⁺.

Particularly preferred bases are K₂CO₃, Na₂SiO₃, NH₄OH, KaPO₄, or KOAc.

The base is preferably used as a saturated alkaline solution in water, wherein the weight ratio of saturated alkaline solution to PU is in the range from by preference 0.5 to 25, preferably 0.5 to 15, more preferably 1 to 10, in particular 2 to 7.

Preferred quaternary ammonium salts have the general structure: R₁ R₂ R₃ R₄ NX
where R₁, R₂, R₃ and R₄ are identical or different hydrocarbon groups selected from alkyl, aryl and/or arylalkyl, wherein R₁ to R₄ are preferably selected such that the sum of the carbon atoms in the quaternary ammonium cation is 6 to 14, preferably 7 to 14, in particular 8 to 13.
X is selected from halide, preferably chloride and/or bromide, hydrogensulfate, alkyl sulfate, preferably methylsulfate or ethylsulfate, carbonate, hydrogencarbonate or carboxylate, preferably acetate or hydroxide.

Very particularly preferred quaternary ammonium salts are tributylmethylammonium chloride, tetrabutylammonium hydrogensulfate, benzyltrimethylammonium chloride, tributylmethylammonium chloride and/or trioctylmethylammonium methylsulfate.

The organic sulfonate containing at least 7 carbon atoms that is likewise employable as catalyst preferably comprises alkyl aryl sulfonates, alpha-olefin sulfonates, petroleum sulfonates and/or naphthalene sulfonates.

Preferred temperatures for the depolymerization are 80° C. to 200° C., preferably 90° C. to 180° C., more preferably 95° C. to 170° C. and in particular 100° C. to 160° C.

Preferred reaction times for the depolymerization are 1 minute to 14 h, by preference 10 minutes to 12 h, preferably 20 minutes to 11 h and in particular 30 minutes to 10 h

The depolymerization is preferably carried out using at least 0.5% by weight of catalyst based on the weight of the polyurethane, preferably 0.5% to 15% by weight, more preferably 1% to 10% by weight, yet more preferably 1% to 8% by weight, yet still more preferably 1% to 7% by weight and in particular 2% to 6% by weight.

A preferred weight ratio of base to polyurethane is within a range from 0.01 to 50, preferably 0.1 to 25, in particular 0.5 to 20.

This related to preferred variant 1 of the depolymerization.

A further particularly preferred variant, referred to here as preferred variant 2, of depolymerization by hydrolysis is described below.

In a further preferred embodiment of the invention, the depolymerization of the polyurethane in step a) is carried out using a base having a pK_{b} at 25° C. of <1, by preference 0.5 to −2, preferably 0.25 to −1.5, in particular 0 to −1, and a catalyst from the group of quaternary ammonium salts containing an ammonium cation having 6 to 14 carbon atoms when the ammonium cation does not comprise a benzyl substituent or else containing an ammonium cation having 6 to 12 carbon atoms when the ammonium cation comprises a benzyl substituent.

Preferred bases are alkali metal hydroxides, alkali metal oxides, alkaline earth metal hydroxides, alkali metal oxides or mixtures thereof. Preferred alkali metals are Na, K or Li or mixtures of the above, in particular Na or K or mixtures thereof; preferred alkaline earth metals are Be, Mg, Ca, Sr or Ba or mixtures thereof, preferably Mg or Ca or mixtures thereof. A very particularly preferred base is NaOH.

Preferred quaternary ammonium salts have the general structure: R₁ R₂ R₃ R₄ NX
where R₁, R₂, R₃ and R₄ are identical or different hydrocarbon groups selected from alkyl, aryl and arylalkyl.
X is selected from halide, preferably chloride and/or bromide, hydrogensulfate, alkyl sulfate, preferably methylsulfate or ethylsulfate, carbonate, hydrogencarbonate, carboxylate, preferably acetate or hydroxide.

Particularly preferred quaternary ammonium salts are benzyltrimethylammonium chloride or tributylmethylammonium chloride.

Preferred temperatures for the depolymerization are 80° C. to 200° C., preferably 90° C. to 180° C., more preferably 95° C. to 170° C. and in particular 100° C. to 160° C.

Preferred reaction times for the depolymerization are 1 minute to 14 h, by preference 10 minutes to 12 h, preferably 20 minutes to 11 h and in particular 30 minutes to 10 h.

The depolymerization is preferably carried out using at least 0.5% by weight of catalyst based on the weight of the polyurethane, preferably 0.5% to 15% by weight, more preferably 1% to 10% by weight, yet more preferably 1% to 8% by weight, yet still more preferably 1% to 7% by weight and in particular 2% to 6% by weight.

A preferred weight ratio of base to polyurethane is in the range from 0.01 to 25, by preference 0.1 to 15, preferably 0.2 to 10, in particular 0.5 to 5.

It is preferable to employ an alkaline solution comprising base and water, wherein the concentration of the base is preferably greater than 5% by weight, by preference 5% to 70% by weight, preferably 5% to 60% by weight, more preferably 10% to 50% by weight, yet more preferably 15% to 40% by weight, in particular 20% to 40% by weight, based on the weight of the alkaline solution.

This related to the preferred variant 2 of the depolymerization.

The PU to be recovered in the PU depolymerization process may be any PU product, in particular comprising a polyurethane foam, preferably rigid PU foam, flexible PU foam, hot-cure flexible PU foam, viscoelastic PU foam, HR PU foam, hypersoft PU foam, semirigid PU foam, thermoformable PU foam and/or integral PU foam.

The described depolymerization of a polyurethane by hydrolysis in the presence of a base and a catalyst selected from the group comprising quaternary ammonium salts containing an ammonium cation comprising 6 to 30 carbon atoms and organic sulfonates containing at least 7 carbon atoms at temperatures preferably below 200° C. thus makes it possible to produce di- and/or polyamines which, optionally after separation of other depolymerization products and reagents used for the depolymerization and optionally preceding purification, may be converted into di- and/or polyisocyanates by phosgenation.

The separation and optionally purification of the relevant di- and/or polyamines may be carried out as follows from the reaction mixture obtained via the depolymerization which may optionally have been pretreated by preceding filtration, separation of the aqueous phase and/or distillation of volatile components: a) by distillation, preferably distillation under reduced pressure in the range from 0.01 mbar to 500 mbar, preferably 0.05 mbar to 350 mbar, more preferably 0.1 mbar to 200 mbar, particularly preferably 0.5 mbar to 100 mbar, or b) by extraction with common organic solvents such as for example toluene, xylene, chlorobenzene, dichlorobenzene, cyclohexane, dichloromethane, tetrahydrofuran, heptane or octane.

Preferred aromatic di- and polyamines obtainable in the process comprise methylenediphenyldiamine (MDA, or else diamines of the diphenylmethane series) as isomers or as an isomer mixture, polymethylenepolyphenylenepolyamine (PMDA, or else polyamines of the diphenylmethane series), mixtures of methylenediphenyldiamine and polymethylenepolyphenylenepolyamine (MDA, or else di- and polyamines of the diphenylmethane series), toluenediamine (TDA) as pure isomers or an isomer mixture of the isomers 2,4-toluenediamine and 2,6-toluenediamine, isomers of xylylenediamine (XDA), isomers of diaminobenzene, 2,6-xylidine, 1,5-naphthylenediamine (1,5-NDA), particularly preferably methylenediphenyldiamine (MDA, or else diamines of the diphenylmethane series) as an isomer mixture, polymethylenepolyphenylenepolyamine (PMDA, or else polyamines of the diphenylmethane series), mixtures of methylenediphenyldiamine and polymethylenepolyphenylenepolyamine (MDA, or else di- and polyamines of the diphenylmethane series), toluenediamine (TDA) as pure isomers or an isomer mixture of the isomers 2,4-toluenediamine and 2,6-toluenediamine, especially preferably toluenediamine (TDA) as a pure isomer or as an isomer mixture of the isomers 2,4-toluenediamine and 2,6-toluenediamine.

Preferred obtainable di- and/or triamines based on aliphatic or cycloaliphatic hydrocarbons having 2 to 18 carbon atoms comprise for example 1,4-diaminobutane, 1,5-diaminopentane, 1,6-diaminohexane (HDA), 1,8-diaminooctane, 1,9-diaminononane, 1,10-diaminodecane, 2,2-dimethyl-1,5-diaminopentane, 2-methyl-1,5-pentanediamine (MPDA), 2,4,4-(or 2,2,4-)trimethyl-1,6-diaminohexane (TMDA), 1,3- and 1,4-diaminocyclohexane, 1-amino-3,3,5-trimethyl-5-aminomethylcyclohexane (IPDA), 2,4-, or 2,6-diamino-1-methylcyclohexane (H6-TDA), 1-amino-1-methyl-4(3)-aminomethylcyclohexane (AMCA), 1,3-(and/or 1,4)-bis(aminomethyl)cyclohexane, bis(aminomethyl)norbornane (NBDA), 4,4′-(and/or 2,4′-)diaminodicyclohexylmethane, (cyclo)aliphatic triamines having up to 22 carbon atoms, for example triaminocyclohexane, tris(aminomethyl)cyclohexane, triaminomethylcyclohexane, 1,8-diamino-4-(aminomethyl)octane, 1,6,1-undecanetriamine, 1,7-diamino-4-(3-aminopropyl)heptane, 1,6-diamino-3-(aminomethyl)hexane and/or 1,3,5-tris(aminomethyl)cyclohexane.

The phosgenation of amines to afford isocyanates is known per se. It may preferably be performed as a gas phase phosgenation of amines previously brought into the gas phase together with gaseous phosgene at temperatures of about 300-400° C. to form the isocyanates in the gaseous state. A phosgene excess is always necessary to prevent the formation of undesired secondary reactions between the isocyanates being formed and the amines used as the starting material. The gas phase phosgenation is typically carried out as a continuous process. The development of the adiabatic gas phase phosgenation, as described for example in EP 1 616 857 A1, makes it possible to save large amounts of energy relative to conventional phosgenation methods. Also possible as well as the gas phase phosgenation is a liquid phase process regime (liquid phase phosgenation), though this has disadvantages due to the large amounts of solvents required.

The present invention thus makes it possible to provide aromatic and/or aliphatic di- and/or polyisocyanates based on polyurethane depolymerization processes as described hereinabove.

Preferred recycled aromatic di- and polyisocyanates obtainable by the process according to the invention include methylendiphenyl diisocyanate (MDI, or else diisocyanates of the diphenylmethane series) as isomers or as an isomer mixture, polymethylenepolyphenylene polyisocyanate (PMDI, or else polyisocyanates of the diphenylmethane series), mixtures of methylenediphenyl diisocyanate and polymethylenepolyphenylene polyisocyanate, toluene diisocyanate (TDI) as pure isomers or a mixture of the isomer 2,4-toluene diisocyanate (2,4-TDI) and 2,6-toluene diisocyanate (2,6-TDI), isomers of xylylene diisocyanate (XDI), isomers of diisocyanatobenzene, 2,6-xylene isocyanate and/or 1,5-naphthylene diisocyanate (1,5-NDI),

particularly preferably methylenediphenyl diisocyanate (MDI, or else diisocyanates of the diphenylmethane series) as isomers or as an isomer mixture, polymethylenepolyphenylene polyisocyanate (PMDI, or else polyisocyanates of the diphenylmethane series), mixtures of methylenediphenyl diisocyanate and polymethylenepolyphenylene polyisocyanate and/or toluene diisocyanate (TDI) as pure isomers or as a mixture of the isomers 2,4-toluene diisocyanate (2,4-TDI) and 2,6-toluene diisocyanate (2,6-TDI), especially preferably toluene diisocyanate (TDI) in isomerically pure form or as a mixture of the isomers 2,4-toluene diisocyanate (2,4-TDI) and 2,6-toluene diisocyanate (2,6-TDI).

Preferred recycled aliphatic or cycloaliphatic di- or polyisocyanates obtainable by the process according to the invention contain 2 to 18 carbon atoms and comprise 1,4-butane diisocyanate, 1,5-pentane diisocyanate, 1,6-hexane diisocyanate (HDI), 1,8-octane diisocyanate, 1,9-nonane diisocyanate, 1,10-decane diisocyanate, 2,2-dimethylpentane-1,5-diisocyanate, 2-methyl-1,5-pentane diisocyanate (MPDI), 2,4,4-(or 2,2,4-)trimethyl-1,6-hexane diisocyanate (TMDI), 1,3- and 1,4-cyclohexane diisocyanate, 1-isocyanato-3,3,5-trimethyl-5-isocyanatomethylcyclohexane (IPDI), 2,4- or 2,6-diisocyanato-1-methylcyclohexane (H6-TDI), 1-isocyanato-1-methyl-4(3)-isocyanatomethylcyclohexane (AMCI), 1,3-(and/or 1,4-)bis(isocyanatomethyl)cyclohexane, bis(isocyanatomethyl)norbornane (NBDI), 4,4′-(and/or 2,4′-)diisocyanatodicyclohexylmethane, (cyclo)aliphatic triisocyanates having up to 22 carbon atoms, for example triisocyanatocyclohexane, tris(isocyanatomethyl)cyclohexane, triisocyanatomethylcyclohexane, 1,8-diisocyanato-4-(Isocyanatomethyl)octane, 1,6,1-undecanetriisocyanate, 1,7-diisocyanato-4-(3-isocyanatopropyl)heptane, 1,6-diisocyanato-3-(isocyanatomethyl)hexane and/or 1,3,5-tris(isocyanatomethyl)cyclohexane.

The resulting di- and/or polyisocyanates may in turn be used to produce new polyurethanes therefrom.

The present invention thus further provides for the use of a di- and/or polyisocyanate obtained by a process according to the invention as described hereinabove for producing polyurethane, in particular PU foam. For the purposes of the present invention, such di- and/or polyisocyanates obtained by a process according to the invention as described hereinabove are also referred to as recycled isocyanates.

The invention also makes it possible to employ large amounts of corresponding recycled isocyanates with only an insignificant reduction, if any, in foam quality compared to a foam made of conventionally produced isocyanates.

In a preferred embodiment of the invention, based on the total isocyanate component, more than 30% by weight, by preference more than 50% by weight, preferably more than 70% by weight, more preferably more than 80% by weight, in particular more than 95% by weight, of recycled isocyanate obtained by a process according to the invention as described hereinabove is present.

The present invention thus further provides a process for producing polyurethane, in particular PU foams, by reacting
(a) at least one polyol component with
(b) at least one isocyanate component
in the presence of
(c) one or more catalysts that catalyse the isocyanate-polyol and/or isocyanate-water and/or isocyanate trimerization reactions,
(d) at least one foam stabilizer and also
(e) optionally one or more chemical or physical blowing agents,
wherein the isocyanate component comprises recycled isocyanate obtained by a process according to the invention as described hereinabove.

In a preferred embodiment of the invention, based on the total isocyanate component, the proportion of recycled isocyanate according to the invention is more than 30% by weight, by preference more than 50% by weight, preferably more than 70% by weight, more preferably more than 80% by weight, in particular more than 95% by weight.

In a further preferred embodiment of the invention, the polyol component likewise comprises recycled polyol, in particular obtained by depolymerization of a polyurethane by hydrolysis in the presence of a base and a catalyst selected from the group consisting of quaternary ammonium salts containing an ammonium cation comprising 6 to 30 carbon atoms and organic sulfonates containing at least 7 carbon atoms as described above.

The process according to the invention using recycled isocyanate makes it possible to provide all known PU foam types. In a preferred embodiment of the invention, the PU foam is a rigid PU foam, a flexible PU foam, a hot-cure flexible PU foam (standard foam), a viscoelastic PU foam, an HR PU foam, a hypersoft PU foam, a semirigid PU foam, a thermoformable PU foam or an integral PU foam, preferably a hot-cure flexible PU foam, HR PU foam, hypersoft PU foam or viscoelastic PU foam. Hot-cure flexible PU foam is most preferred.

In a preferred embodiment of the invention, in particular for producing molded foams and highly elastic flexible PU foams, the isocyanate component employed is preferably toluene diisocyanate (TDI) as an isomer mixture of 2,4- and 2,6-toluene diisocyanate and/or methylenediphenyl diisocyanate (MDI) as an isomer mixture of 4,4′-, 2,4′- and 2,2′-methylenediphenyl diisocyanate and/or polyphenyipolymethylene polyisocyanate (crude MDI or polymeric MDI).

It is particularly preferable to employ TDI in an isomer ratio of 80 to 20 (2,4-TDI to 2,6-TDI) and/or methylenediphenyl diisocyanate (MDI) as an isomer mixture of 4,4′-, 2,4′- and 2,2′-methylenediphenyl diisocyanate and/or polyphenylpolymethylene polyisocyanate (crude MDI or polymeric MDI).

In a further preferred embodiment of the invention which relates to the production of hot-cure flexible foams (standard foams), the isocyanate component employed is preferably toluene diisocyanate (TDI) as an isomer mixture of 2,4- and 2,6-toluene diisocyanate. It is particularly preferable to employ TDI in an isomer ratio of 80 to 20 (2,4-TDI to 2,6-TDI).

A further preferred embodiment of the invention is the production of viscoelastic foams (also known as visco foams). For viscoelastic polyurethane foams, the isocyanate component employed is preferably toluene diisocyanate (TDI) as an isomer mixture of 2,4- and 2,6-toluene diisocyanate and/or methylenediphenyl diisocyanate (MDI) as an isomer mixture of 4,4′-, 2,4′- and 2,2′-methylenediphenyl diisocyanate and/or polyphenylpolymethylene polyisocyanates (crude MDI or polymeric MDI). It is particularly preferable to employ TDI in an isomer ratio of 80 to 20 (2,4-TDI to 2,6-TDI) and/or in an isomer ratio of 65 to 35 (2,4-TDI to 2,6-TDI) and/or methylenediphenyl diisocyanate as a mixture of 4,4′-, 2,4′- and 2,2′-methylenediphenyl diisocyanates and polyphenylpolymethylene polyisocyanate. The aromatic polyisocyanates mentioned may be employed individually or else in the form of mixtures thereof. To produce viscoelastic polyurethane foams it is preferable to employ mixtures of TDI in an isomer ratio of 80 to 20 (2,4-TDI to 2,6-TDI) and TDI in an isomer ratio of 65 to 35 (2,4-TDI to 2,6-TDI) or mixtures of TDI in an isomer ratio of 80 to 20 (2,4-TDI to 2,6-TDI) and methylenediphenyl diisocyanate as a mixture of 4,4′-, 2,4′- and 2,2′-methylenediphenyl diisocyanates and polyphenylpolymethylene polyisocyanate.

The production of the PU foams may in principle be carried out in the customary manner and as described in the prior art. It is well known to those skilled in the art. A comprehensive overview is found in, for example, G. Oertel, Polyurethane Handbook, 2nd edition, Hanser/Gardner Publications Inc., Cincinnati, Ohio, 1994, pp. 177-247. Further details of the starting materials, catalysts and auxiliaries and additives that may be used can be found for example in Kunststoffhandbuch [Plastics Handbook], volume 7, Polyurethane [Polyurethanes], Carl-Hanser-Verlag Munich, 1st edition 1966, 2nd edition 1983 and 3rd edition 1993.

In a further preferred embodiment of the invention, production of the PU foams according to the invention is carried out using
f) water,
g) one or more organic solvents,
h) one or more stabilizers against oxidative degradation, in particular antioxidants,
i) one or more flame retardants, and/or
j) one or more further additives, preferably selected from the group of surfactants, biocides, dyes, pigments, fillers, antistatic additives, crosslinkers, chain extenders, cell openers, fragrances, cell expanders, plasticizers, hardening promoters, aldehyde scavengers, additives for resistance of PU foams to hydrolysis, compatibilizers (emulsifiers), adhesion promoters, hydrophobization additives, flame-lamination additives, additives for preventing cold flow, additives that reduce compression set, additives for adjusting the glass transition temperature, temperature-controlling additives and/or odour-reducers.

The present invention further provides a composition suitable for producing polyurethane foam comprising at least one polyol component, at least one isocyanate component, catalyst, foam stabilizer, blowing agent, optionally auxiliaries, wherein the isocyanate component comprises recycled isocyanate as described hereinabove.

Preferred optional auxiliaries comprise surfactants, biocides, dyes, pigments, fillers, antistatic additives, crosslinkers, chain extenders, cell openers, as described for example in EP 2998333A1, fragrances, cell expanders, as described for example in EP 2986661B1, plasticizers, hardening promoters, additives for preventing cold flow, as described for example in DE 2507161C3, WO 2017029054A1, aldehyde scavengers, as described for example in WO 2021/013607A1, additives for resistance of PU foams to hydrolysis, as described for example in US 2015/0148438A1, compatibilizers (emulsifiers), adhesion promoters, hydrophobization additives, flame-lamination additives, as described for example in EP 2292677A1, additives that reduce compression set, additives for adjusting the glass transition temperature, temperature-controlling additives and/or odour-reducers.

The compounds employed according to the invention, the production thereof, the use of the compounds for producing the PU foams and the PU foams themselves are hereinbelow described by way of example without any intention to limit the invention to these exemplary embodiments. Where ranges, general formulas or compound classes are specified below, these are intended to include not only the corresponding ranges or groups of compounds that are explicitly mentioned but also all subranges and subgroups of compounds that can be obtained by removing individual values (ranges) or compounds. Where documents are cited in the context of the present description, their content shall fully form part of the disclosure content of the present invention, particularly in respect of the matters referred to. Where figures are hereinbelow reported in percent, these percentages are percentages by weight unless otherwise stated. Average values specified hereinbelow are number averages unless otherwise stated. Where properties of a material are referred to hereinbelow, for example viscosities or the like, these are the properties of the material at 25° C. unless otherwise stated. Where chemical (empirical) formulas are used in the present invention, the stated indices may be not only absolute numbers but also average values. For polymeric compounds, the indices preferably represent average values.

The process according to the invention makes it possible to obtain any PU foams. Preferred PU foams are for the purposes of the present invention flexible PU foams and rigid PU foams. Flexible PU foams and rigid PU foams are established technical terms. The known and fundamental difference between flexible foams and rigid foams is that flexible foam shows elastic behaviour and hence deformation is reversible. By contrast, rigid foam undergoes permanent deformation. Various foam subgroups preferred in the context of the invention are described in more detail hereinbelow.

In the context of the present invention, rigid polyurethane foam is in particular to be understood as meaning a foam according to DIN 7726: 1982-05 that has a compressive strength according to DIN 53421:1984-06 of advantageously ≥20 kPa, by preference ≥80 kPa, preferably ≥100 kPa, more preferably ≥150 kPa, particularly preferably ≥180 kPa. In addition, the rigid polyurethane foam according to DIN EN ISO 4590:2016-12 advantageously has a closed-cell content of greater than 50%, preferably greater than 80% and more preferably greater than 90%. Rigid PU foams are used mostly for insulation purposes.

Flexible PU foams are elastic and deformable and usually open-celled. This means that air can escape easily on compression. The umbrella term “flexible PU foam” here includes in particular the following foam types known to those skilled in the art, namely hot-cure flexible PU foam (standard PU foam), cold-cure PU foam, (also highly elastic or high-resilience foam), hypersoft PU foam, viscoelastic flexible PU foam and ester-type PU foams (from polyester polyols). The different flexible PU foam types are more particularly elucidated and delimited from one another hereinbelow.

The crucial difference between a hot-cure flexible PU foam and a cold-cure PU foam lies in the different mechanical properties. It is possible to differentiate between flexible hot-cure PU foams and flexible cold-cure PU foams in particular via rebound resilience, also known as ball rebound (BR) or resilience. A method for determining the rebound resilience is described for example in DIN EN ISO 8307:2008-03. In this method, a steel ball having a fixed mass is allowed to fall from a defined height onto the test specimen and the height of the rebound as a % of the drop height is then measured. Hot-cure flexible PU foams have rebound values of preferably 1% to not more than 50%. The height of the rebound in the case of cold-cure flexible PU foams is preferably within the range >50%. The high rebound resilience of cold-cure flexible PU foams results from a relatively irregular cell size distribution. A further mechanical criterion is the sag or comfort factor. Here, a foam specimen is compressed according to DIN EN ISO 2439:2009-05 and the ratio of compressive stress at 65% and 25% compression is measured. Hot-cure flexible PU foams have a comfort factor of preferably <2.5. In the case of cold-cure flexible PU foams, the comfort factor is preferably >2.5. The production of cold-cure flexible PU foams employs in particular polyether polyols that are highly reactive towards isocyanates and have a high proportion of primary hydroxyl groups and number-average molar masses >4000 g/mol. By contrast, hot-cure flexible PU foams generally employ predominantly less reactive polyols having secondary OH groups and an average molar mass of <4000 g/mol. As well as cold-cure slabstock PU foams, moulded cold-cure PU foams, which are used for example in automotive seat cushioning, represent a core use of cold-cure PU foams.

Likewise preferred according to the invention are hypersoft PU foams, which represent a subcategory of flexible PU foams. Hypersoft PU foams have compressive stresses determined to DIN EN ISO 3386-1:1997+A1:2010 of preferably <2.0 kPa and exhibit indentation hardnesses determined to DIN EN ISO 2439:2009-05 of preferably <80 N. Hypersoft PU foams may be produced by various known processes: through use of a so-called hypersoft polyol in combination with so-called standard polyols and/or through a special production process in which carbon dioxide is added during the foaming process. As a consequence of a pronounced open-cell structure, hypersoft PU foams have high air permeability, promote moisture transfer in application products and help avoid heat buildup. The hypersoft polyols used for production of hypersoft PU foams have the particular feature of a very high proportion of primary OH groups of more than 60%.

A special class of flexible PU foams is that of viscoelastic PU foams (visco foams), which are likewise preferred according to the invention. These are also known as “memory foam” and feature both a low rebound resilience according to DIN EN ISO 8307:2008-03 of preferably <15% and a slow, gradual recovery after compression (recovery time preferably 2-13 s). In contrast to hot-cure flexible PU foams and cold-cure flexible PU foams, which have a glass transition temperature of less than −32° C., for viscoelastic PU foams the glass transition temperature is preferably shifted to within a range from −20 to +15° C. Such “structural viscoelasticity” in the case of open-cell viscoelastic flexible PU foams, which is based essentially on the glass transition temperature of the polymer (also referred to as chemical visco foams), should be distinguished from a pneumatic effect. In the latter case, the cell structure is relatively closed (low porosity). The low air permeability means that the air flows back in only gradually after compression, which results in slowed recovery (also referred to as pneumatic visco foams). In many cases the two effects are combined in a visco foam. PU visco foams are highly prized on account of their energy- and sound-absorbing properties.

A class of PU foams that is particularly important for applications in the automotive sector and has properties in between those of rigid and flexible foams is that of semirigid (semiflexible) PU foams. These too are preferred according to the invention. Like most PU foam systems, semiflexible foam systems also make use of the isocyanate/water reaction and of the CO₂ evolved as a blowing agent for foam formation. The rebound resilience is generally lower than that of classical flexible foams, especially cold-cure foams. Semiflexible foams have higher hardness than conventional flexible foams. A characteristic feature of semiflexible foams is their high open-cell content (preferably >90% of cells). The densities of semiflexible foams can be significantly higher than those of flexible and rigid foams.

The polyol components employed are preferably one or more polyols having two or more OH groups. Preferred employable polyols include all polyether polyols and polyester polyols used for production of polyurethane systems, in particular polyurethane foam systems.

Polyether polyols are obtainable for example by reacting polyfunctional alcohols or amines with alkylene oxides. Polyester polyols are based preferably on esters of polybasic carboxylic acids with polyhydric alcohols (usually glycols). The polybasic carboxylic acids can either be aliphatic (for example adipic acid) or aromatic (for example phthalic acid or terephthalic acid).

An important class of optionally employable polyols obtainable from natural oils such as palm oil or soybean oil are known as “natural oil-based polyols” (NOPs) and can be obtained on the basis of renewable raw materials. NOPs are of increasing interest for more sustainable production of PU foams in view of the long-term limits on the availability of fossil resources—oil, coal and gas—and against the background of rising crude oil prices and have already been described many times in the production of polyurethane foams (WO 2005/033167; US 2006/0293400, WO 2006/094227, WO 2004/096882, US 2002/0103091, WO 2006/116456 and EP 1678232) A number of these polyols are now commercially available from various manufacturers (WO 2004/020497, US 2006/0229375, WO 2009/058367). Depending on the base raw material (e.g. soybean oil, palm oil or castor oil) and subsequent processing, polyols having different profiles of properties are obtained. A distinction may essentially be made between two groups: a) polyols based on renewable raw materials that are modified such that they may be used to an extent of 100% in the production of polyurethanes (WO2004/020497, US2006/0229375); b) polyols based on renewable raw materials that on account of their processing and properties are able to replace the petrochemical-based polyol only up to a certain proportion (WO 2009/058367) The production of polyurethane foams from recycled polyols together with NOPs represents a preferred form of application of the invention.

A further class of optionally employable polyols comprises polyols obtained as prepolymers by reaction of polyol with isocyanate in a molar ratio of 100:1 to 5:1, preferably 50:1 to 10:1.

Yet another class of optionally employable polyols comprises what are known as filled polyols (polymer polyols). These contain dispersed solid organic fillers up to a solids content of 40% by weight or more. Employable polyols include for example and inter alia:

SAN polyols: These are highly reactive polyols containing a dispersed copolymer based on styrene-acrylonitrile (SAN).

PUD polyols: These are highly reactive polyols containing polyurea particles in dispersed form.

PIPA polyols: These are highly reactive polyols containing polyurethane particles in dispersed form, produced for example by in-situ reaction of an isocyanate with an alkanolamine in a conventional polyol.

The solids content in the optional filled polyols, which depending on the application may preferably be between 5% and >40% by weight based on the polyol, is responsible for improved cell opening, with the result that the polyol becomes controllably foamable, especially with TDI, and no shrinkage of the foams occurs. The solids content thus acts as an essential processing aid. A further function is to control the hardness via the solids content, since higher solids contents result in a higher hardness of the foam.

Formulations comprising polyols that contain solids have markedly reduced inherent stability and therefore tend to require not only chemical stabilization through the crosslinking reaction but also additional physical stabilization.

Other optionally employable polyols are those known as cell-opener polyols. These are polyether polyols having a high ethylene oxide content, specifically a content preferably of at least 40% by weight, in particular of 50% to 100% by weight, based on the content of alkylene oxide.

A ratio of isocyanate component to polyol component that is preferred in the context of the present invention, and is expressed as an index, is within a range from 10 to 1000, preferably 40 to 350. This index describes the ratio of the amount of isocyanate actually used to the amount of isocyanate theoretically required for a stoichiometric ratio of isocyanate groups to isocyanate-reactive groups (e.g. OH groups, NH groups), multiplied by 100. An index of 100 represents a molar ratio of reactive groups of 1:1.

The isocyanate component necessarily contains recycled isocyanate obtained by a process according to the invention as described hereinabove. In the context of the present invention. the term recycled isocyanates encompasses di- and/or polyisocyanates obtained by a process according to the invention as described hereinabove.

In a preferred embodiment of the invention, the proportion of recycled isocyanate based on the total isocyanate component employed is more than 30% by weight, by preference more than 50% by weight, preferably more than 70% by weight, more preferably more than 80% by weight, in particular more than 95% by weight.

Isocyanate components employed are preferably one or more isocyanates comprising two or more isocyanate functions, wherein the isocyanate component comprises recycled diisocyanate obtainable according to the invention. In the process according to the invention, the optionally additionally employable isocyanates may be any isocyanates, in particular the aliphatic, cycloaliphatic, araliphatic and preferably aromatic polyfunctional isocyanates known per se. Suitable isocyanates for the purposes of the present invention have two or more isocyanate functions.

Suitable isocyanates for the purposes of the present invention are preferably any polyfunctional organic isocyanates, for example diphenylmethane diisocyanate (MDI), toluene diisocyanate (TDI), hexamethylene diisocyanate (HMDI) and/or isophorone diisocyanate (IPDI). Preference is likewise given to using the mixture known as “polymeric MDI” (“crude MOI” or polyphenyl polymethylene polyisocyanate), composed of MDI and analogues with a higher level of condensation having an average functionality of 2 to 4.

Particular preference is given to using diphenylmethane 2,4′-diisocyanate and/or diphenylmethane 2,2′-diisocyanate and/or polyphenyl polymethylene polyisocyanate (crude MDI) and/or toluene 2,4-diisocyanate and/or toluene 2,6-diisocyanate or mixtures thereof.

MDI prepolymers are preferably also particularly suitable. Examples of particularly suitable isocyanates are detailed for example in EP 1712578, EP 1161474, WO 00/58383, US 2007/0072951, EP 1678232 and WO 2005/085310, which are hereby fully incorporated by reference.

Suitable catalysts for possible use in the process of the invention for producing PU foams are preferably substances that catalyse the gel reaction (isocyanate-polyol), the blowing reaction (isocyanate-water) or the di- or trimerization of the isocyanate.

In a preferred embodiment of the invention, the catalyst employed is selected from triethylenediamine, 1,4-diazabicyclo[2.2.2]octane-2-methanol, diethanolamine, N-[2-[2-(dimethylamino)ethoxy]ethyl]-N-methyl-1,3-propanediamine, 2-[[2-(2-(dimethylamino)ethoxy)ethyl]methylamino]ethanol, 1,1′-[(3-{bis[3-(dimethylamino)propyl]amino}propyl)imino]dipropan-2-ol, [3-(dimethylamino)propyl]urea, 1,3-bis[3-(dimethylamino)propyl]urea and/or amine catalysts having the general structure (1a) and/or the structure (1b):

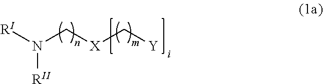

X comprises oxygen, nitrogen, hydroxyl, amines having the structure (NR^{III} or NR^{III}R^{IV}) or urea groups (N(R^{V})C(O)N(R^{VI}) or N(R^{VII})C(O)NR^{VI}R^{VII}).
Y comprises amines NR^{VIII}R^{IX} or ethers OR^{IX},
R^{I,II} comprise identical or different, linear or cyclic, aliphatic or aromatic hydrocarbons having 1-8 carbon atoms that are optionally functionalized with an OH group and/or comprise hydrogen,
R^{III-IX} comprise identical or different, linear or cyclic, aliphatic or aromatic hydrocarbons having 1-8 carbon atoms that are optionally functionalized with an OH group, an NH group or an NH₂ group and/or comprise hydrogen,
m=0 to 4, preferably 2 or 3,
n=2 to 6, preferably 2 or 3,
i=0 to 3, preferably 0-2,

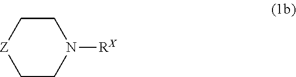

R^{X} comprises identical or different radicals consisting of hydrogen and/or linear, branched or cyclic, aliphatic or aromatic hydrocarbons having 1-18 carbon atoms, which may be substituted with 0-1 hydroxyl groups and 0-1 NH₂ groups,
Z comprises oxygen, N—R^{X} or CH₂.

A further class of suitable catalysts that may be used with preference in the process according to the invention are metal compounds of the metals Sn, Bi, Zn, Al or K, in particular Sn, Zn or Bi. The metal compounds can be divided into the subgroups of organometallic compounds, organometallic salts, organic metal salts and inorganic metal salts, which are explained hereinbelow.

The expression “metal-organic or organometallic compounds” encompasses for the purposes of this invention in particular the use of metal compounds having a direct carbon-metal bond, here also referred to as metal organyls (e.g. tin organyls) or organometallic/organometal compounds (e.g. organotin compounds). The expression “organometallic or metal-organic salts” encompasses for the purposes of this invention in particular the use of metal-organic or organometallic compounds having salt character, i.e. ionic compounds in which either the anion or cation is metal-organic in nature (e.g. organotin oxides, organotin chlorides or organotin carboxylates). The expression “organic metal salts” encompasses for the purposes of this invention in particular the use of metal compounds that do not have any direct carbon-metal bond and are at the same time metal salts in which either the anion or the cation is an organic compound (e.g. tin(II) carboxylates). The expression “inorganic metal salts” encompasses for the purposes of this invention in particular the use of metal compounds or of metal salts in which neither the anion nor the cation is an organic compound, e g metal chlorides (e.g. tin(II) chloride).

Organic and organometallic metal salts that are suitable for use contain preferably alkoxide, mercaptate or carboxylate anions, such as acetate, 2-ethylhexanoate, octanoate, isononanoate, decanoate, neodecanoate, ricinoleate, laurate and/or oleate, particularly preferably 2-ethylhexanoate, ricinoleate, neodecanoate or isononanoate.

As a general rule, metal-containing catalysts that are suitable for use are preferably selected such that they do not have any troublesome intrinsic odour and are essentially toxicologically safe, and such that the resulting polyurethane systems, especially polyurethane foams, have the lowest possible degree of catalyst-related emissions.

It may be preferable to combine one or more metal compounds with one or more amine catalysts of formula (1a) and/or (1b).

In the inventive production of polyurethane foams, it may be preferable to exclude the use of organometallic salts, for example of dibutyltin dilaurate.

Suitable usage amounts of catalyst in the process according to the invention depend on the type of catalyst and are preferably in the range from 0.01 to 5 pphp (=parts by weight based on 100 parts by weight of polyol) or from 0.1 to 10 pphp in the case of potassium salts.

Suitable water contents in the process of the invention depend on whether or not physical blowing agents are used in addition to water. In the case of purely water-blown foams, values range from preferably 1 to 20 pphp; when other blowing agents are additionally used, the amount of water used is reduced to typically e.g. 0 or e.g 0.1 to 5 pphp. To achieve high foam densities, it is preferable that neither water nor any other blowing agent is used.

Physical blowing agents that are optionally suitable for use for the purposes of the present invention are gases, for example liquefied CO₂, and volatile liquids, for example hydrocarbons having 4 or 5 carbon atoms, preferably cyclo-, iso- and n-pentane, hydrofluorocarbons, preferably HFC 245fa, HFC 134a and HFC 365mfc, but also olefinic hydrofluorocarbons such as HHO 1233zd or HHO1336mzzZ, hydrochlorofluorocarbons, preferably HCFC 141b, oxygen-containing compounds such as methyl formate and dimethoxymethane, or chlorinated hydrocarbons, preferably dichloromethane and 1,2-dichloroethane. Suitable blowing agents further include ketones (e.g. acetone) or aldehydes (e.g. methylal).

In addition to or in place of water and any physical blowing agents, the additive composition of the invention may also include other chemical blowing agents that react with isocyanates with gas evolution, examples being formic acid, carbamates or carbonates.

Foam stabilizers (referred to as stabilizers for the purposes of the invention) that can be used include the substances mentioned in the prior art. The compositions of the invention may advantageously contain one or more stabilizers. They are in particular silicon compounds containing carbon atoms, preferably selected from polysiloxanes, polydimethylsiloxanes, organomodified polysiloxanes, polyether-modified polysiloxanes and polyether-polysiloxane copolymers. Preferred silicon compounds are described by formula (1c)

[R¹₂R²SiO_{1/2}]ₐ[R¹₃SiO_{1/2}]_{b}[R¹₂SiO_{2/2}]_{c}[R¹R²SiO_{2/2}]_{d}[R³SiO_{3/2}]ₑ[SiO_{4/2}]_{f}G_{g} Formula (1c):

where
a=0 to 12, preferably 0 to 10, more preferably 0 to 8,
b=0 to 8, preferably 0 to 6, more preferably 0 to 2,
c=0 to 250, preferably 1 to 200, more preferably 1.5 to 150,
d=0 to 40, preferably 0 to 30, more preferably 0 to 20,
e=0 to 10, preferably 0 to 8, more preferably 0 to 6,
f=0 to 5, preferably 0 to 3, more preferably 0,
g=0 to 3, preferably 0 to 2.5, more preferably 0 to 2
where:

$$a + b + c + d + e + f + g > 3 ,$$

$$a + b \geq 2 ,$$

G=independently identical or different radicals consisting of

$${( {O}_{1 / 2} )}_{n} {SiR}_{m}^{1} - {CH}_{2} {CHR}^{5} - {R}^{4} - {CHR}^{5} {CH}_{2} - {{SiR}_{m}^{1} ( {O}_{1 / 2} )}_{n} ,$$

$${( {O}_{1 / 2} )}_{n} {SiR}_{m}^{1} - {CH}_{2} {CHR}^{5} - {R}^{4} - {CR}^{5} = {CH}_{2} ,$$

$${( {O}_{1 / 2} )}_{n} {SiR}_{m}^{1} - {CH}_{2} {CHR}^{5} - {R}^{4} - {CR}^{5} = {CR}^{5} - {CH}_{3} ,$$

R⁴=independently identical or different divalent organic radicals, preferably divalent organic radicals consisting of 1 to 50 carbon atoms, optionally interrupted by ether, ester or amide groups and optionally functionalized with OH groups, or (—SiR¹₂O—)ₓSiR¹₂ groups, more preferably identical or different divalent organic radicals consisting of 2 to 30 carbon atoms, optionally interrupted by ether, ester or amide groups and optionally functionalized with OH groups, or (—SiR¹₂O—)ₓSiR¹₂ groups,
x=1 to 50, preferably 1 to 25, more preferably 1 to 10,
R⁵=independently identical or different alkyl radicals consisting of 1 to 16 carbon atoms, aryl radicals having 6 to 16 carbon atoms or hydrogen, preferably from the group of alkyl radicals having 1 to 6 carbon atoms or aryl radicals having 6 to 10 carbon atoms or hydrogen, more preferably methyl or hydrogen,
where:

$$n = 1 or 2 ,$$

$$m = 1 or 2 ,$$

$$n + m = 3 ,$$

R¹=identical or different radicals selected from the groups of saturated or unsaturated alkyl radicals having 1 to 16 carbon atoms or aryl radicals having 6 to 16 carbon atoms or hydrogen or —OR₆, preferably methyl, ethyl, octyl, dodecyl, phenyl or hydrogen, more preferably methyl or phenyl,
R²=independently identical or different polyethers obtainable by the polymerization of ethylene oxide and/or propylene oxide and/or other alkylene oxides such as butylene oxide or styrene oxide of general formula (2) or an organic radical corresponding to formula (3)

—(R⁷)ₕ—O—[C₂H₄O]ᵢ—[C₃H₈O]ⱼ—[CR⁸₂CR⁸₂O]ₖ—R⁹, (2)

—Oₕ—R¹⁰, (3)
where
h=0 or 1,
R⁷=divalent organic radical, preferably divalent organic alkyl or aryl radical optionally substituted with —OR₃, more preferably a divalent organic radical of type CₚH₂ₚ,
i=0 to 150, preferably 0 to 100, more preferably 0 to 80.
j=0 to 150, preferably 0 to 100, more preferably 0 to 80,
k=0 to 80, preferably 0 to 40, more preferably 0,
p=1-18, preferably 1-10, more preferably 3 or 4,
where

$$i + j + k \geq 3 ,$$

R³=identical or different radicals selected from the group of saturated or unsaturated alkyl radicals potentially substituted with heteroatoms, preferably identical or different radicals selected from the group of saturated or unsaturated alkyl radicals having 1 to 16 carbon atoms or aryl radicals having 6-16 carbon atoms potentially substituted with halogen atoms, more preferably methyl, vinyl, chloropropyl or phenyl,
R⁶=identical or different radicals selected from the group of saturated or unsaturated alkyl radicals having 1 to 16 carbon atoms or aryl radicals having 6 to 16 carbon atoms or hydrogen, preferably saturated or unsaturated alkyl radicals having 1 to 8 carbon atoms or hydrogen, more preferably methyl, ethyl, isopropyl or hydrogen,
R⁸=identical or different radicals selected from the group of alkyl radicals having 1 to 18 carbon atoms, potentially substituted with ether functions and potentially substituted with heteroatoms such as halogen atoms, aryl radicals having 6-18 carbon atoms, potentially substituted with ether functions, or hydrogen, preferably alkyl radicals having 1 to 12 carbon atoms, potentially substituted with ether functions and potentially substituted with heteroatoms such as halogen atoms or aryl radicals having 6-12 carbon atoms, potentially substituted with ether functions, or hydrogen, more preferably methyl, ethyl, benzyl or hydrogen,
R⁹=identical or different radicals selected from the group hydrogen, alkyl, —C(O)—R¹¹, —C(O)O—R¹¹ or —C(O)NHR¹¹, saturated or unsaturated, optionally substituted with heteroatoms, preferably hydrogen or alkyl radicals having 1 to 8 carbon atoms or acetyl, more preferably hydrogen, acetyl, methyl or butyl,
R¹⁰=identical or different radicals selected from the group of saturated or unsaturated alkyl radicals or aryl radicals, potentially substituted with one or more OH, ether, epoxide, ester, amine and/or halogen substituents, preferably saturated or unsaturated alkyl radicals having 1 to 18 carbon atoms or aryl radicals having 6-18 carbon atoms, optionally substituted with one or more OH, ether, epoxide, ester, amine and/or halogen substituents, more preferably saturated or unsaturated alkyl radicals having 1 to 18 carbon atoms or aryl radicals having 6-18 carbon atoms substituted with at least one OH, ether, epoxide, ester, amine and/or halogen substituent,
R¹¹=identical or different radicals selected from the group of alkyl radicals having 1 to 16 carbon atoms or aryl radicals having 6 to 16 carbon atoms, preferably saturated or unsaturated alkyl radicals having 1 to 8 carbon atoms or aryl radicals having 6 to 12 carbon atoms, more preferably methyl, ethyl, butyl or phenyl.

The foam stabilizers of formula (1c) may preferably be used in organic solvents such as for example dipropylene glycol, polyether alcohols or polyether diols blended in PU systems.

In the case of mixtures of stabilizers of formula (1c), it is additionally preferably possible to use a compatibilizer. This compatibilizer may be selected from the group of aliphatic or aromatic hydrocarbons, more preferably aliphatic polyethers or polyesters.

Employable silicon compounds having one or more carbon atoms preferably include the substances mentioned in the prior art. Preference is given to using those silicon compounds that are particularly suitable for the particular type of foam. Suitable siloxanes are described for example in the following documents: EP 0839852, EP 1544235, DE 102004001408, WO 2005/118668, US 2007/0072951, DE 2533074, EP 1537159, EP 533202, U.S. Pat. No. 3,933,695, EP 0780414, DE 4239054, DE 4229402, EP 867465. The silicon compounds may be produced as described in the prior art. Suitable examples are described e.g. in U.S. Pat. No. 4,147,847, EP 0493836 and U.S. Pat. No. 4,855,379.

From 0.00001 to 20 parts by mass of foam stabilizers, in particular silicon compounds, per 100 parts by mass of polyol components may preferably be used.

Optional additives used may be all substances known from the prior art that are used in the production of polyurethanes, in particular of polyurethane foams, examples being blowing agents, preferably water for formation of CO₂, and, if necessary, further physical blowing agents, flame retardants, buffer substances, surfactants, biocides, dyes, pigments, fillers, antistatic additives, crosslinkers, chain extenders, cell openers, as described for example in EP 2998333A1, nucleating agents, thickeners, fragrances, cell expanders, as described for example in EP 2986661B1, plasticizers, hardening promoters, additives for preventing cold flow, as described for example in DE 2507161C3, WO 2017029054A1, aldehyde scavengers, as described for example in WO 2021/013607A1, additives for resistance of PU foams to hydrolysis, as described for example in US 2015/0148438A1, compatibilizers (emulsifiers), adhesion promoters, hydrophobization additives, flame-lamination additives, as described for example in EP 2292677B1, additives that reduce compression set, odour-reducers and/or additional catalytically active substances, in particular as defined above.

Optionally employable crosslinkers and optionally employable chain extenders are low-molecular-weight polyfunctional compounds that are reactive towards isocyanates. Examples of suitable compounds are hydroxyl- or amine-terminated substances such as glycerol, neopentyl glycol, dipropylene glycol, sugar compounds, 2-methylpropane-1,3-diol, triethanolamine (TEOA), diethanolamine (DEOA) and trimethylolpropane. Employable crosslinkers likewise include polyethoxylated and/or polypropoxylated glycerol or sugar compounds having a number-average molecular weight below 1500 g/mol. The optional use concentration is preferably between 0.1 and 5 parts based on 100 parts of polyol, but can also deviate therefrom depending on the formulation. When crude MDI is used in in-situ foaming, it likewise takes on a crosslinking function. The content of low-molecular-weight crosslinkers can therefore be accordingly reduced as the amount of crude MDI increases.

Suitable optional stabilizers against oxidative degradation, so-called antioxidants, preferably include all commonly used free-radical scavengers, peroxide scavengers, UV absorbers, light stabilizers, complexing agents for metal ion contaminants (metal deactivators). Preferably employable compounds are compounds of the following substance classes or substance classes containing the following functional groups: 2-(2′-hydroxyphenyl)benzotriazoles, 2-hydroxybenzophenones, benzoic acids and benzoates, phenols, in particular comprising tert-butyl and/or methyl substituents on the aromatic entity, benzofuranones, diarylamines, triazines, 2,2,6,6-tetramethylpiperidines, hydroxylamines, alkyl and aryl phosphites, sulfides, zinc carboxylates, diketones.

Suitable optional flame retardants for the purposes of the present invention are all substances considered suitable for this purpose according to the prior art. Examples of preferred flame retardants are liquid organophosphorus compounds such as halogen-free organophosphates, for example triethyl phosphate (TEP), halogenated phosphates, e.g. tris(1-chloro-2-propyl) phosphate (TCPP), tris(1,3-dichloroisopropyl) phosphate (TDCPP) and tris(2-chloroethyl) phosphate (TCEP), and organic phosphonates, for example dimethyl methanephosphonate (DMMP), dimethyl propanephosphonate (DMPP), or oligomeric ethyl-ethylene phosphates or solids such as ammonium polyphosphate (APP) and red phosphorus. Suitable flame retardants further include halogenated compounds, for example halogenated polyols, and also solids such as expandable graphite and melamine.

The process according to the invention makes it possible to produce polyurethane foams of any kind. The term polyurethane is for the purposes of the present invention to be understood in particular as a generic term for a polymer produced from di- or polyisocyanates and polyols or other isocyanate-reactive species, such as for example amines, wherein the urethane linkage is not necessarily the sole or predominating linkage type. Polyisocyanurates and polyureas are also expressly included.

The production according to the invention of polyurethane foams can be carried out by any processes familiar to those skilled in the art, for example by manual mixing or preferably with the aid of high-pressure or low-pressure foaming machines. The process of the invention may be executed continuously or batchwise. Batchwise execution of the process is preferable in the production of moulded foams, refrigerators, footwear soles or panels. A continuous process is preferable for producing insulation panels, metal composite elements, slabs or in spraying methods.

The invention further provides a polyurethane foam, preferably rigid PU foam, flexible PU foam, hot-cure flexible PU foam (standard foam), viscoelastic PU foam, HR PU foam, hypersoft PU foam, semirigid PU foam, thermoformable PU foam or integral PU foam, preferably hot-cure flexible PU foam, HR PU foam, hypersoft PU foam or viscoelastic PU foam, produced by a process of the invention as described hereinabove. Hot-cure flexible PU foams are most preferred.

A very particularly preferred flexible polyurethane foam has for the purposes of the present invention in particular the following composition:

| Component | Parts by weight (pphp) |
|---|---|
| Polyol | 100 |
| (Amine) catalyst | 0.01 to 5 |
| Tin catalyst | 0 to 5, preferably 0.001 to 2 |
| Siloxane | 0.1 to 15, preferably 0.2 to 7 |
| Water | 0 to <15, preferably |
| Further blowing agents | 0 to 130 |
| Flame retardant | 0 to 70 |
| Fillers | 0 to 150 |
| Further additives | 0 to 20 |
| Isocyanate comprising recycled isocyanate | |
| Isocyanate index: | greater than 50 |

The polyurethane foams according to the invention may be used for example as refrigerator insulation, insulation panels, sandwich elements, pipe insulation, spray foam, 1- and 1.5-component can foam (a 1.5-component can foam is a foam that is produced by destroying a container in the can), imitation wood, modelling foam, packaging foam, mattresses, furniture cushioning, automotive seat cushioning, headrests, instrument panels, automotive interior trim, automotive headlining, sound absorption material, steering wheels, shoe soles, carpet backing foam, filter foam, sealing foam, sealants, adhesives, binders, lacquers or as coating, or for producing corresponding products. This corresponds to a further subject matter of the invention.

### EXAMPLES

### Production of the Inventive Recycled Toluene Diisocyanate

The inventive recycled toluene diisocyanate was obtained by hydrolysis of polyurethane in the presence of a saturated K₂CO₃ solution and tetrabutylammonium hydrogensulfate as catalyst and subsequent phosgenation of the isolated toluenediamine mixture.

A reactor from Parr (Parr Instrumental Company) equipped with a PTFE inner container and a mechanical stirrer was filled with 25 g of compressed foam pieces (approx. 1 cm×1 cm). The polyurethane foam employed was produced according to formulation 1 specified hereinbelow. 75 g of saturated K₂CO₃ solution were then added (pK_{b} 3.67 at 25° C.). The catalyst tetrabutylmethylammonium hydrogensulfate was then added in an amount of 5% by weight based on the mass of the reaction mixture. The reactor was closed and the reaction mixture was heated to an internal temperature of 150° C. for 14 hours. At the end of the 14 hours, heating was stopped and the reaction mixture was cooled to room temperature. After opening the reactor, the reaction mixture was transferred to a round-bottomed flask. The water was removed by rotary evaporation and the residual reaction mixture extracted with cyclohexane and subsequently filtered. The solid filtered off was extracted with toluene and the obtained extraction solution was dried. Removal of the toluene by rotary evaporation afforded the toluenediamine as an isomer mixture. For conversion into the isocyanate, 24 g of the toluenediamine were dissolved in 1.2 L of toluene. 250 ml of a 0.157 molar solution of triphosgene in toluene were subsequently added. After complete addition, the reaction mixture was heated to 110° C. and stirred under reflux for 2 h at this temperature. The reaction mixture was then cooled to room temperature and filtered. Removal of the solvent by rotary evaporation afforded the toluene diisocyanate isomer mixture. The process was repeated so as to provide a sufficiently large amount of recycled toluene diisocyanate for the foaming experiments.

### Production of Flexible PU Foams

To test the recycled toluene diisocyanate in terms of its foaming properties and its influence on physical foam properties, the following formulation was used for production of hot-cure flexible foam. For example, 1.0 part (1.0 pphp) of a component refers to 1 g of this substance per 100 g of polyol.

### Table 1: Formulation for Production of Hot-Cure Flexible PU Foams

| Formulation 1 | parts by mass (pphp) | |
|---|---|---|
| Polyol¹⁾ | 100 | pphp |
| Water | 4.00 | pphp |
| KOSMOS ® T9²⁾ | 0.20 | pphp |
| DABCO ® DMEA³⁾ | 0.15 | pphp |
| TEGOSTAB ® BF2370⁴⁾ | 1.0 | pphp |

| | |
|---|---|
| Toluene diisocyanate T 80⁵⁾ | variable, index of 105 |

¹⁾Polyol: Standard Polyether Polyol Arcol ® 1104 available from Covestro; this is a glycerol-based polyether polyol having an OH number of 56 mg KOH/g and a number-average molar mass of 3000 g/mol.
²⁾KOSMOS ® T9, obtainable from Evonik Industries: tin(II) salt of 2-ethylhexanoic acid.
³⁾DABCO ® DMEA: dimethylethanolamine, available from Evonik Industries. Amine catalyst for production of polyurethane foams
⁴⁾Polyether-modified polysiloxane, available from Evonik Industries.
⁵⁾Toluene diisocyanate: Conventional toluene diisocyanate Desmodur ® T 80 available from Covestro; this is a toluene diisocyanate T 80 (80% 2,4-isomer, 20% 2,6-isomer) having a viscosity of 3 mPa · s, 48% NCO and a functionality of 2 or inventive recycled toluene isocyanate.

### General Procedure for Production of Hot-Cure Flexible PU Foams

The polyurethane foams were produced in the laboratory as so-called handmade foams. The production of the foams was carried out at 22° C. and an air pressure of 762 mmHg as specified below. The polyurethane foams according to formulation I were in each case produced using 100 g of polyol. The other formulation constituents were adjusted accordingly. This means, for example, that 1.0 part (1.0 pphp) of a component refers to 1 g of said substance per 100 g of polyol.

For the foams according to formulation I, a paper cup was initially charged with the tin catalyst tin(II) 2-ethylhexanoate, polyol, the water, the amine catalysts and the respective foam stabilizer, and the contents were mixed with a disc stirrer at 1000 rpm for 60 s. After the first stirring, the isocyanate was added and incorporated with the same stirrer at 2500 rpm for 7 s and the reaction then immediately transferred to a paper-lined box (19 cm×19 cm base area and 19 cm height). After the foam had been poured in, it rose up in the foaming box. In the ideal case, the foam blew off on reaching the maximum rise height and then receded slightly. This opened the cell membranes of the foam bubbles and an open-pore cell structure of the foam was obtained.

To assess the properties, the characteristic parameters described in the following section were determined.

### Performance Tests

The foams produced were assessed on the basis of the following physical properties
a) Settling of the foam at the end of the rise phase (=fall-back).
   The settling, or the further rise, is calculated as the difference in the foam height immediately after blow-off and after 3 minutes after foam blow-off. The foam height is measured in this case at the maximum in the centre of the foam crest by means of a needle secured to a centimetre scale. A positive value here describes the settling of the foam after blow-off; a negative value correspondingly describes the further rise of the foam.
b) Foam height is the height of the freely risen foam formed after 3 minutes. The foam height is reported in centimetres (cm).
c) Rise time
   The period of time between the end of mixing of the reaction components and the blow-off of the polyurethane foam. The rise time is reported in seconds (s).
d) Porosity
   The air permeability of the foam was determined based on DIN EN ISO 4638 1993-07 by a dynamic pressure measurement on the foam. The measured dynamic pressure was reported in mm water column, lower dynamic pressure values being characteristic of a more open foam. The values were measured within a range from 0 to 300 mm water column. The dynamic pressure was measured by means of an apparatus comprising a nitrogen source, reducing valve with pressure gauge, flow-regulating screw, wash bottle, flowmeter, T-piece, applicator nozzle and a graduated glass tube filled with water. The applicator nozzle has an edge length of 100×100 mm, a weight of 800 g, an internal diameter at the outlet opening of 5 mm, an internal diameter at the lower applicator ring of 20 mm and an external diameter at the lower applicator ring of 30 mm.
   The measurement is carried out by setting the nitrogen inlet pressure to 1 bar by adjusting the reducing valve and setting the flow rate to 480 I/h. The amount of water in the graduated glass tube is set so that no pressure difference builds up and none can be read off. For the measurement on a test specimen having dimensions of 150×150×50 mm, the applicator nozzle is applied to the corners of the test specimen, flush with the edges, and also once to the (estimated) centre of the test specimen (in each case on the side having the greatest surface area). The result is read off when a constant dynamic pressure has been established. The evaluation is based on the calculated average of the five measurements obtained.
e) Number of cells per cm (cell count): This is determined visually on a cut surface (measured to DIN EN 15702:2009-04).

### Results of the Foaming Operations

The inventive recycled toluene diisocyanate is tested in comparison with the conventional toluene diisocyanate T 80 in formulation I, table 1. The results of the performance tests for the use of the various isocyanates are reported in table 2.

TABLE 2
Foaming results for the hot-cure flexible PU foams produced
according to formulation 1, table 1, using the inventive
recycled diisocyanate and the conventional toluene
diisocyanate Desmodur T 80 from Covestro.

| Foam specimen | #1 | #2 |
|---|---|---|
| Desmodur ® T 80 | 50 pphp | 35 pphp |
| Recycled toluene | | 15 pphp |
| diisocyanate (inventive) | | |
| Index | 105 | 105 |
| Rise time (s) | 115 | 114 |
| Foam height (cm) | 19.9 | 19.1 |
| Settling (cm) | 0.0 | 0.2 |
| Cell count (cm⁻¹) | 12 | 11-12 |
| Porosity (mm water column) | 14 | 16 |
| Remarks | Standard | Standard |
| | foam | foam |

The results in table 2 show that the inventive recycled toluene diisocyanate may be employed as the isocyanate component to an extent of 30%, wherein comparable foaming behavior analogous to that seen when using 100% of the conventional toluene diisocyanate Desmodur® T 80 is observed. Especially the rise time remains almost unchanged. The foam height of foam #2 is only slightly below that of reference foam #1 with Desmodur® T80. Likewise the obtained foam bodies #1 and #2 exhibit comparable physical foam properties in terms of porosity and cell number.

1. A process for producing aromatic and/or aliphatic di- and/or polyisocyanates, comprising:
a) depolymerizing a polyurethane by hydrolysis in the presence of a base and a catalyst selected from the group consisting of quaternary ammonium salts containing an ammonium cation comprising 6 to 30 carbon atoms and organic sulfonates containing at least 7 carbon atoms to produce di- and/or polyamines,
b) separating the di- and/or polyamines obtained from a) from a reaction mixture by extraction, distillation and/or other separation processes,
c) phosgenating the di- and/or polyamines obtained from b) to afford di- and/or polyisocyanates, wherein di- and/or polyamines not deriving from a) may optionally also be added in c).

2. The process according to claim 1, wherein the resulting di- and/or polyisocyanates comprise aromatic and/or aliphatic di- and/or polyisocyanates.

3. The process according to claim 1, wherein the depolymerization of the polyurethane in a) is effected using a base having a pK_{b} at 25° C. of 1 to 10 and also a catalyst selected from the group comprising quaternary ammonium salts containing an ammonium cation comprising 6 to 30 carbon atoms and organic sulfonates containing at least 7 carbon atoms.

4. The process according to claim 1, wherein the depolymerization of the polyurethane in a) is carried out using a base having a pK_{b} at 25° C. of <1 and a catalyst selected from the group consisting of quaternary ammonium salts containing an ammonium cation having 6 to 14 carbon atoms when the ammonium cation does not comprise a benzyl substituent and quaternary ammonium salts containing an ammonium cation having 6 to 12 carbon atoms when the ammonium cation does comprise a benzyl substituent.

5. The process according to claim 1, wherein the polyurethane to be depolymerized in a) comprises a polyurethane foam.

6. A process for producing polyurethane, comprising: further reacting the di- and/or polyisocyanate obtainable by a process according to claim 1.

7. A process for producing polyurethane, comprising:
reacting
(a) at least one polyol component with
(b) at least one isocyanate component
in the presence of
(c) one or more catalysts that catalyse the isocyanate-polyol and/or isocyanate-water and/or isocyanate trimerization reactions,
(d) at least one foam stabilizer, and also
(e) optionally one or more chemical or physical blowing agents,
wherein the at least one isocyanate component comprises recycled isocyanate obtained by the process according to claim 1.

8. The process according to claim 7, wherein the at least one isocyanate component contains more than 30% by weight of recycled isocyanate based on the total isocyanate component.

9. The process according to claim 7, wherein the at least one polyol component comprises a recycled polyol.

10. The process according to claim 7, wherein the at least one foam stabilizer is selected from the group consisting of silicon compounds containing carbon atoms and mixtures of two or more of these compounds:
[R¹₂R²SiO_{1/2}]ₐ[R¹₃SiO_{1/2}]_{b}[R¹₂SiO_{2/2}]_{c}[R¹R²SiO_{2/2}]_{d}[R³SiO_{3/2}]ₑ[SiO_{4/2}]_{f}G_{g} Formula (1c):
where
a=0 to 12,
b=0 to 8,
c=0 to 250,
d=0 to 40,
e=0 to 10,
f=0 to 5,
g=0 to 3,
where:
$$a + b + c + d + e + f + g > 3 ,$$
$$a + b \geq 2 ,$$
G-independently identical or different radicals consisting of
$${( {O}_{1 / 2} )}_{n} {SiR}_{m}^{1} - {CH}_{2} {CHR}^{5} - {R}^{4} - {CHR}^{5} {CH}_{2} - {{SiR}_{m}^{1} ( {O}_{1 / 2} )}_{n} ,$$
$$( {O}_{1 / 2} ) {n SiR}_{m}^{1} - {CH}_{2} {CHR}^{5} - {R}^{4} - {CR}^{5} = {CH}_{2} ,$$
$$( {O}_{1 / 2} ) {n SiR}_{m}^{1} - {CH}_{2} {CHR}^{5} - {R}^{4} - {CR}^{5} = {CR}^{5} - {CH}_{3} ,$$
R⁴=independently identical or different divalent organic radicals, optionally interrupted by ether, ester or amide groups and optionally functionalized with OH groups, or (—SiR¹₂O—)ₓSiR¹₂ groups,
x=1 to 50,
R⁵=independently identical or different alkyl radicals consisting of 1 to 16 carbon atoms, aryl radicals having 6 to 16 carbon atoms or hydrogen,
where:
$$n = 1 or 2 ,$$
$$m = 1 or 2 ,$$
$$n + m = 3 ,$$
R¹=identical or different radicals selected from the group of saturated or unsaturated alkyl radicals having 1 to 16 carbon atoms or aryl radicals having 6 to 16 carbon atoms or hydrogen or —OR⁶,
R²=independently identical or different polyethers obtainable by the polymerization of ethylene oxide and/or propylene oxide and/or other alkylene oxides having the general formula (2) or an organic radical corresponding to formula (3)
—(R⁷)ₕ—O—[C₂H₄O]ᵢ—[C₃H₈O]ⱼ—[CR⁸₂CR⁸₂O]ₖ—R⁹, (2)
—Oₕ—R¹⁰, (3)
where
h=0 or 1,
R⁷=divalent organic radical,
i=0 to 150,
j=0 to 150,
k=0 to 80,
p=1-18,
where
$$i + j + k \geq 3 ,$$
R³=identical or different radicals selected from the group of saturated or unsaturated alkyl radicals potentially substituted with heteroatoms,
R⁶=identical or different radicals selected from the group of saturated or unsaturated alkyl radicals having 1 to 16 carbon atoms or aryl radicals having 6 to 16 carbon atoms or hydrogen,
R⁸=identical or different radicals selected from the group of alkyl radicals having 1 to 18 carbon atoms, potentially substituted with ether functions and potentially substituted with heteroatoms, aryl radicals having 6-18 carbon atoms, potentially substituted with ether functions, or hydrogen,
R⁹=identical or different radicals selected from the group hydrogen, alkyl, —C(O)—R¹¹, —C(O)O—R¹¹ or —C(O)NHR¹¹, saturated or unsaturated, optionally substituted with heteroatoms,
R¹⁰=identical or different radicals selected from the group of saturated or unsaturated alkyl radicals or aryl radicals, potentially substituted with one or more OH, ether, epoxide, ester, amine and/or halogen substituents,
R¹¹=identical or different radicals selected from the group of alkyl radicals having 1 to 16 carbon atoms or aryl radicals having 6 to 16 carbon atoms.

11. The process according to claim 7, wherein the catalyst for producing PU foam is selected from triethylenediamine, 1,4-diazabicyclo[2.2.2]octane-2-methanol, diethanolamine, N-[2-[2-(dimethylamino)ethoxy]ethyl]-N-methyl-1,3-propanediamine, 2-[[2-(2-(dimethylamino)ethoxy)ethyl]methylamino]ethanol, 1,1′-[(3-{bis[3-(dimethylamino)propyl]amino}propyl)imino]dipropan-2-ol, [3-(dimethylamino)propyl]urea, 1,3-bis[3-(dimethylamino)propyl]urea and/or amine catalysts having the general structure (1a) and/or the structure (1b): 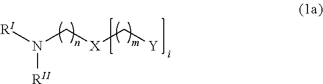
X comprises oxygen, nitrogen, hydroxyl, amines having the structure (NR^{III} or NR^{III}R^{IV}) or urea groups (N(R^{V})C(O)N(R^{VI}) or N(R^{VII})C(O)NR^{VI}R^{VII}),
Y comprises amines NR^{VIII}R^{IX} or ethers OR^{IX},
R^{I,II} comprise identical or different, linear or cyclic, aliphatic or aromatic hydrocarbons having 1-8 carbon atoms that are optionally functionalized with an OH group and/or comprise hydrogen,
R^{III-IX} comprise identical or different, linear or cyclic, aliphatic or aromatic hydrocarbons having 1-8 carbon atoms that are optionally functionalized with an OH group, an NH group or an NH₂ group and/or comprise hydrogen,
m=0 to 4,
n=2 to 6,
i=0 to 3, 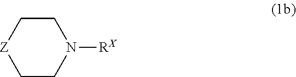
R^{X} comprises identical or different radicals consisting of hydrogen and/or linear, branched or cyclic, aliphatic or aromatic hydrocarbons having 1-18 carbon atoms, which may be substituted with 0-1 hydroxyl groups and 0-1 NH₂ groups,
Z comprises oxygen, N—R^{X} or CH₂,
and/or
metal compounds including organometallic metal salts, organic metal salts, inorganic metal salts and also organometallic compounds of the metals Sn, Bi, Zn, Al or K.

12. A composition suitable for producing polyurethane foam, comprising:
at least one polyol component, at least one isocyanate component, a catalyst, a foam stabilizer, a blowing agent and optionally auxiliaries, wherein the at least one isocyanate component comprises recycled isocyanate obtained by a process according to claim 1.

13. A polyurethane foam, wherein it is obtained by a process according to claim 7.

14. A process for manufacturing goods, comprising: installing the PU foam according to claim 13 as at least one selected from the group consisting of refrigerator insulation, insulation panels, sandwich elements, pipe insulation, spray foam, 1-component can foam, 1.5-component can foam, imitation wood, modelling foam, packaging foam, mattresses, furniture cushioning, automotive seat cushioning, headrests, instrument panels, automotive interior trim, automotive headlining, sound absorption material, steering wheels, shoe soles, carpet backing foam, filter foam, sealing foam, sealants, adhesives, coatings, and for production of corresponding products.

15. The process according to claim 1, wherein the resulting di- and/or polyisocyanates comprise at least one aromatic and/or aliphatic di- and/or polyisocyanate selected from the group consisting of methylenediphenyl diisocyanate, polymethylene polyphenylene polyisocyanate, toluene diisocyanate, and isophorone diisocyanate.

16. The process according to claim 1, wherein the resulting di- and/or polyisocyanates comprise toluene diisocyanate.

17. The process according to claim 1, wherein the polyurethane to be depolymerized in a) comprises at least one polyurethane foam selected from the group consisting of rigid PU foam, flexible PU foam, hot-cure flexible PU foam, viscoelastic PU foam, HR PU foam, hypersoft PU foam, semirigid PU foam, thermoformable PU foam and integral PU foam.

18. A process for producing PU foams, comprising:
reacting
(a) at least one polyol component with
(b) at least one isocyanate component
in the presence of
(c) one or more catalysts that catalyse the isocyanate-polyol and/or isocyanate-water and/or isocyanate trimerization reactions,
(d) at least one foam stabilizer, and also
(e) optionally one or more chemical or physical blowing agents,
wherein the at least one isocyanate component comprises recycled isocyanate obtained by the process according to claim 1.

19. The process according to claim 7, wherein the at least one polyol component comprises a recycled polyol obtained by depolymerization of a polyurethane by hydrolysis in the presence of a base and a catalyst selected from the group consisting of quaternary ammonium salts containing an ammonium cation comprising 6 to 30 carbon atoms and organic sulfonates containing at least 7 carbon atoms.
